(19) United States
(12) Patent Application Publication
(10) Pub. No.: US 2025/0356962 A1
(43) Pub. Date: Nov. 20, 2025

(54) SYSTEM AND METHOD FOR GENERATING SYNTHETIC PATIENT DATA AND SIMULATING CLINICAL STUDIES

(71) Applicant: IQVIA Inc.

(72) Inventors: Kallol Chaudhuri, Parsippany, NJ (US); Guanhao Wei, Parsippany, NJ (US); Yue Wang, Shanghai City (CN); Russell Reeve, Apex, NC (US); Adrian McKemey, Parsippany, NJ (US); Sirish Kumar Konda, Parsippany, NJ (US); Yunlong Wang, Malvern, PA (US)

(21) Appl. No.: 18/022,598

(22) PCT Filed: Jan. 20, 2023
(86) PCT No.: PCT/CN2023/073356

§ 371 Date: Feb. 22, 2023

(51) Int. Cl. G16H 10/20; G06N 3/0464; G06N 3/08; G16H 50/20; G16H 50/70

(52) U.S. Cl. CPC G16H 10/20; G06N 3/0464; G06N 3/08; G16H 50/20; G16H 50/70

(57) ABSTRACT

Methods, systems, and apparatus for generating synthetic patient data and simulating clinical studies. In one aspect, a method includes obtaining a disease of interest for an in silico clinical study and obtaining historic patient data associated with the disease of interest. The historic patient data includes patient attributes for each patient. The method includes, based on the patient attributes, generating synthetic patient data. The synthetic patient data reproduce statistical properties of the historic patient data. The method includes applying the synthetic patient data to the in silico clinical study configured to predict a clinical study outcome and providing, based on the predicted clinical study outcome, feedback data that specify one or more parameters used in generating the synthetic patient data.

### TECHNICAL FIELD

The present disclosure is directed towards generating synthetic patient data and simulating clinical studies using the synthetically generated patient data.

### BACKGROUND

Clinical studies, e.g., clinical trials, post-market studies, safety studies, and studies of diseases, carry uncertainties in terms of treatment response, disease progression, and adverse events. These uncertainties are attributed to failure in clinical studies in resulting in approved treatments for diseases and better understanding of diseases. Careful selection of patient groups to be included in the clinical studies can help minimize these uncertainties in clinical studies.

### SUMMARY

This specification describes techniques for generating synthetic patient data and simulating clinical studies with the synthetic patient data. The synthetic patient data enables simulating clinical studies with varied study populations, thereby predicting clinical study outcomes and improving success of the clinical study. The synthetic patient data are particularly useful when there are not enough patient data that meet a sample size requirement for a well-powered statistical test. In addition, the generated synthetic patient data can be repurposed for other similar clinical studies, leading to improved prediction of outcomes. Simulated clinical studies are referred to as in silico clinical studies. The in silico clinical studies reduce costs associated with designing and carrying out clinical studies while increasing their respective success rates.

In an aspect, a computer-implemented method includes obtaining, by one or more processors, a disease of interest for an in silico clinical study. The computer-implemented method includes obtaining, by the one or more processors, historic patient data associated with the disease of interest. The historic patient data includes, for each patient, a plurality of patient attributes. The computer-implemented method includes, by the one or more processors and based on the plurality of patient attributes, generating synthetic patient data. The synthetic patient data reproduce statistical properties of the historic patient data. The computer-implemented method includes, by the one or more processors, applying the in silico clinical study to the synthetic patient data. The in silico clinical study is configured to predict a clinical study outcome. The computer-implemented method includes, by the one or more processors and based on the predicted clinical study outcome, providing feedback data that specify one or more parameters used in generating the synthetic patient data.

Embodiments can include one or any combination of two or more of the following features.

The computer-implemented method further includes determining an inclusion and exclusion criterion; identifying a subset of the historic patient data that meet the inclusion and exclusion criterion; and generating synthetic patient data that correspond to the subset of the historic patient data.

The plurality of patient attributes includes biomarkers of the disease of interest.

Generating the synthetic patient data includes: determining a multivariate correlation structure among the plurality of patient attributes in the historic patient data; and generating the synthetic patient data that maintain the multivariate correlation structure.

The computer-implemented method further includes validating, based on comparing a first multivariate correlation structure in the historic patient data and a second multivariate correlation structure in the synthetic patient data, the synthetic patient data.

Comparing the first multivariate correlation structure and the second multivariate correlation structure includes determining a Cramer test p-value and a Bhattacharyya coefficient. The Cramer test p-value and the Bhattacharyya coefficient are corrected for multiple hypothesis.

The clinical study outcome includes one or more of a treatment response, a disease progression, and an adverse event.

The one or more parameters used in generating synthetic patient data include a control sample size, a case sample size, and an algorithm to generate the synthetic patient data.

The computer-implemented method further includes providing, on a user interface, the clinical study outcome stratified by the plurality of patient attributes. The user interface includes user selectable elements to adjust a plurality of inclusion and exclusion criteria.

The historic patient data include a first set of patient data and a second set of patient data. A plurality of patients in the synthetic patient data corresponding to the first set of patient data receives a treatment in the in silico clinical study.

Applying the in silico clinical study to the synthetic patient data includes applying, to the synthetic patient data, a machine learning model trained to predict the clinical study outcome. The clinical study outcome includes a treatment response, a disease progression, and an adverse event; and obtaining the predicted clinical study outcome.

The computer-implemented method further includes training the machine learning model on a plurality of training patient data, each of the plurality of training patient data is labeled with a clinical outcome. The machine learning model uses convolutional neural networks.

The computer-implemented method further includes combining the historic patient data and the synthetic patient data and applying the in silico clinical study to the combined historic patient data and the synthetic patient data.

Providing feedback data that specify the one or more parameters used in generating the synthetic patient data includes identifying one or more biomarkers different from the patient attributes included in the historic patient data; obtaining second historic patient data that include the one or more biomarkers; and providing the second historic patient data. The second historic patient data are used to generate second synthetic patient data.

In an aspect, a system includes one or more processors and one or more storage devices storing instructions that are operable, when executed by the one or more processors, to cause the one or more processors to perform operations including: obtaining, by the one or more processors, a disease of interest for an in silico clinical study; and obtaining, by the one or more processors, historic patient data associated with the disease of interest. The historic patient data includes, for each patient, a plurality of patient attributes. The operations include, by the one or more processors and based on the plurality of patient attributes, generating synthetic patient data. The synthetic patient data reproduce statistical properties of the historic patient data. The operations include, by the one or more processors, applying the synthetic patient data to the in silico clinical study. The in silico clinical study is configured to predict a clinical study outcome. The operations include, by the one or more processors and based on the predicted clinical study outcome, providing feedback data that specify one or more parameters used in generating the synthetic patient data.

In an aspect, a non-transitory computer-readable medium, including software instructions, that when executed by a computer, cause the computer to execute operations including obtaining, by the computer, a disease of interest for an in silico clinical study and obtaining, by the computer, historic patient data associated with the disease of interest. The historic patient data includes, for each patient, a plurality of patient attributes. The operations include, by the computer and based on the plurality of patient attributes, generating synthetic patient data. The synthetic patient data reproduce statistical properties of the historic patient data. The operations include, by the computer, applying the synthetic patient data to the in silico clinical study. The in silico clinical study is configured to predict a clinical study outcome. The operations include, by the computer and based on the predicted clinical study outcome, providing feedback data that specify one or more parameters used in generating the synthetic patient data.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is a block diagram of an example system for generating synthetic patient data and simulating clinical studies.

FIG. 2A shows an example of distributions of original patient data.

FIG. 2B shows an example of distributions of synthetic patient data.

FIGS. 3A-3B shows an example user interface.

FIG. 4 is a flowchart of example process for generating synthetic patient data and simulating clinical studies using the generated synthetic patient data.

FIG. 5 is a block diagram of example system components that can be used to implement a system for generating synthetic patient data and simulating clinical studies.

### DETAILED DESCRIPTION

According to an aspect of the present disclosure, systems and methods for generating synthetic patient data and simulating clinical studies are disclosed. Synthetic patient data retain statistical properties of historic (original) patient data. For example, distributions of variables, such as sex, age, and blood test measurements, are retained in the synthetic patient data. In addition, the synthetic data do not include identifiable information, e.g., a patient's name, a date of birth, and an email address. In some implementations, the synthetic data are validated to ensure their quality, e.g., by comparing statistical properties between the historic patient data and the synthetic patient data. In simulating clinical studies, also referred to as in silico clinical studies, the synthetic patient data can be used, instead of or in addition to the historic patient data. The in silico clinical study predicts a clinical outcome, e.g., a disease progression, in a given study population defined by the patient data used for the in silico clinical study. Based on the predicted clinical outcome, the synthetic patient data are refined, e.g., by regenerating by tuning parameters used in generating the synthetic patient data. The predicted clinical outcome informs designers of clinical studies, e.g., researchers, recommendations on the study population, e.g., the number of case and control patients and patient attributes. For example, the predicted clinical outcome may show controlling a certain medical history, e.g., a tobacco usage, is essential, and thus the recommendation may include the study population having no tobacco usage.

The system and methods of the present disclosure can have one or more of the following advantages. First, the synthetic patient data described here meet privacy requirements of handling identifiable information often found in patient data such as electronic medical records. Because of privacy requirements imposed on patient data, usage of such data is often limited. Second, the synthetic patient data enable predicting outcomes of a clinical study. For example, even if the patient data can be utilized to simulate a clinical study, there might not be enough samples within the patient data. The synthetic patient data increases effective sample sizes and thus improve the statistical power. As yet another example, when the patient data are heavily imbalanced (e.g., many female samples and few male samples), the synthetic patient data can offset the imbalanced patient data. Third, an in silico clinical study reduces computational burdens in iterative process of simulating a clinical study. In particular, feedback data provided by the in silico clinical study reduces the number of iterations involved in the simulation and saves computational power and resources. Fourth, the in silico clinical study has a practical application for increasing success rates of developing a new treatment for diseases. Not only patients enrolled for a clinical study, but also prospective patients receive benefits for the new treatment powered by the in silico clinical study. Fifth, the outcome of the in silico clinical study can lead to discovery of new biomarkers. For example, correlation structures among patient attributes reveal which patient attributes are significantly correlated with the outcome of the in silico clinical study, e.g., a particular molecular measurement associated with a survival rate of a cardiovascular disease. The new biomarkers can provide another source of feedback data to refining the synthetic patient data and can lead to increased prediction accuracies in terms of treatment response (e.g., which patients respond to a given treatment), disease progression (e.g., how does the disease progress based on receiving a given treatment for a case group and not receiving a given treatment for a control group), and adverse events (e.g., side effects, survival rate), among others.

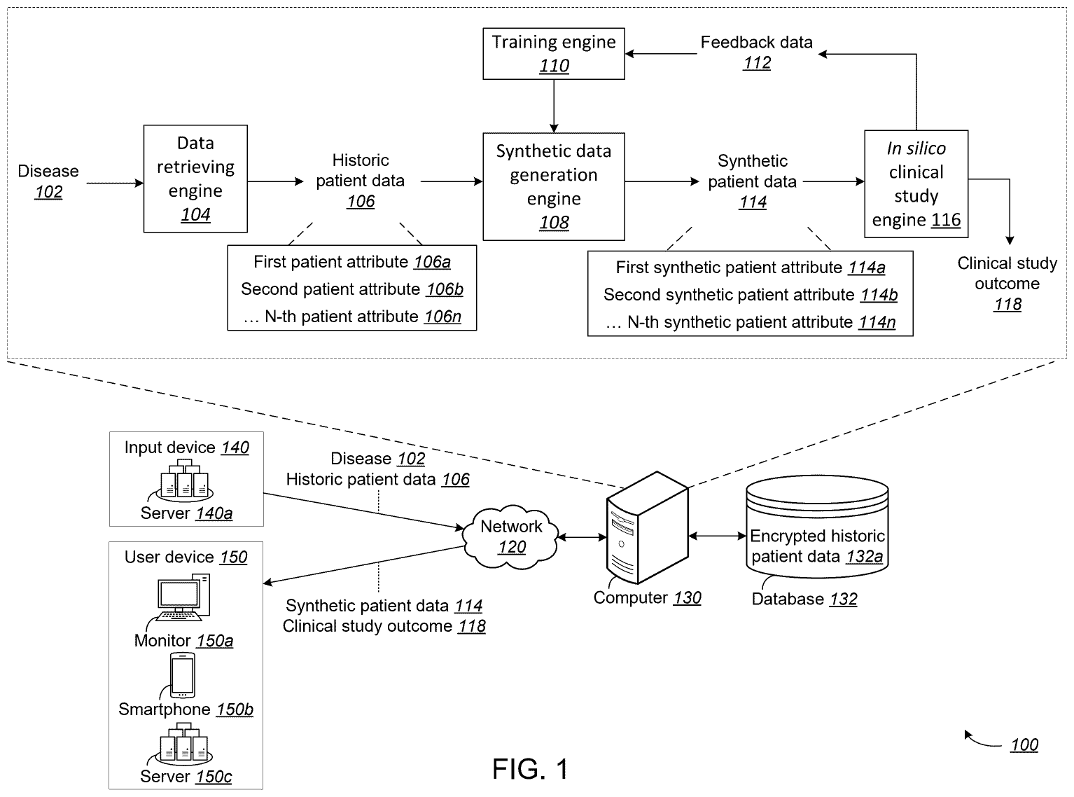

FIG. 1 is a block diagram of an example of a system 100 that generates synthetic patient data and simulate clinical studies. The system 100 includes an input device 140, a network 120, and one or more computers 130 (e.g., one or more local or cloud-based processors). The computer 130 can include a data retrieving engine, a synthetic data generation engine 108, a training engine 110, and an in silico clinical study engine 116. In some implementations, the computer 130 is a server. While not shown, the system 100 can include a separate training engine that trains the in silico clinical study engine 116. For purposes of the present disclosure, an “engine” can include one or more software modules, one or more hardware modules, or a combination of one or more software modules and one or more hardware modules. In some implementations, one or more computers are dedicated to a particular engine. In some implementations, multiple engines can be installed and running on the same computer or computers.

The input device 140 is a device that is configured to obtain an identification of a disease of interest 102 and/or historic patient data 106 (collectively referred to as input data), a device that is configured to provide the disease 102 and/or historic patient data 106 to another device across the network 120, or any suitable combination thereof. The disease of interest 102 refers to data indicative of a disease of interest, e.g., a user input of a name of the disease or a text file including a name of the disease. For example, the input device 140 can include a server 140a that is configured to obtain the input data, e.g., electronic health records of patients regarding patients' medical histories. In some implementations, the server 140a can obtain the historic patient data 106, e.g., by accessing a database of medical records, and transmit the historic patient data 106 to another device such as the computer 130 across the network 120. In some implementations, the server 140a can obtain the disease 102 and use the disease 102 to look up the historic patient data 106 in a database. The obtained input data can be transmitted to the computer 130 via the network 120. The network 120 can include one or more of a wired Ethernet network, a wired optical network, a wireless WiFi network, a LAN, a WAN, a Bluetooth network, a cellular network, the Internet, or other suitable network, or any combination thereof. In some implementations, the server 140a and the computer 130 are the same.

The computer 130 is configured to obtain data for the disease 102 from the input device 140 such as the server 140a. In some implementations, a user inputs the disease 102 via a user interface on a user device, e.g., a portable computing device, associated with the user. The disease 102 represents the disease of interest for a particular clinical study. For example, for a treatment being developed for lowering cholesterol, the disease 102 is hyperlipidemia. In some implementations, the input device 140 infers the disease 102 based on either the treatment or the clinical study without a user input of the disease 102. In some implementations, the historic patient data 106 can be retrieved without the disease 102. In this case, the computer 130 can identify a subset of the historic patient data 106 that are relevant for a particular clinical study.

The data retrieving engine 104 is configured to obtain data for the disease 102 and generate the historic patient data 106. The historic patient data 106 includes one or more patient attributes (a first patient attribute 106a, a second patient attribute 106b, . . . . N-th patient attribute 106n). For example, the data retrieving engine 104 accesses the database 132, e.g., a local database or a cloud-based database connected to the computer 130, that stores the encrypted historic patient data 132a and obtains a subset of the encrypted historic patient data 132a that meet the inclusion and exclusion criteria for the disease 102. In some implementations, the inclusion and exclusion criteria of the study population is specified by the user, e.g., via a user interface. In some implementations, the inclusion and exclusion criteria of the study population is automatically determined based on the disease 102. The inclusion and exclusion criteria may include the presence of the disease 102 or other related diseases (e.g., a disease known to have a comorbidity to the disease 102). The historic patient data 106 may include identifiable information; in this case, the data retrieving engine 104 removes such identifiable information.

In some implementations, the data retrieving engine 104 accesses multiple databases and standardizes the obtained historic patient data. This may be necessary because different database may save patient data in different formats and units. The data retrieving engine 104 can estimate missing values based on available patient data. For example, when a particular patient's blood pressure is missing, the data retrieving engine 104 estimates the blood pressure based on data from other similar patients. The data retrieving engine 104 can convert non-standardized patient attributes in the historic patient data, e.g., by using a standardized format and unit across data.

The synthetic data generation engine 108 is configured to receive the historic patient data 106 and generate synthetic patient data 114. The synthetic data generation engine 108 processes the historic patient data 106 such that the synthetic patient data 114 closely reproduce statistical properties, e.g., a correlation structure among patient attributes and medians of patient attributes, of the historic patient data 106. The synthetic patient data 114 includes one or more synthetic patient attributes (a first synthetic patient attribute 114a, a second synthetic patient attribute 114b, . . . , N-th synthetic patient attribute 114n). The synthetic patient data 114 need not to include all corresponding patient attributes to the historic patient data 106. In some implementations, the synthetic patient data 114 include a subset of patient attributes.

The synthetic data generation engine 108 can be trained by the training engine 110. The training engine 110 generates one or more synthetic data generation models, each model using a different algorithm from k-nearest neighbors to multidimensional correlation generative (MCG) methods. The k-nearest neighbors method generates a synthetic sample based on k number of sampled original data. The MCG method generates a synthetic sample in a way that a correlation structure among patient attributes is preserved. The training engine 110 can also indicate the sample size, e.g., the number of patients for case and control groups. The synthetic data generation engine 108 uses these parameters, from an algorithmic choice to a sample size, in generating the synthetic patient data 114.

The in silico clinical study engine 116 is configured to receive the synthetic patient data 114 and generate clinical study outcome 118 and feedback data 112. The clinical study outcome includes one or more of a treatment response, a disease progression, and an adverse event. The in silico clinical study engine 116 invokes a machine learning model configured to predict the clinical study outcome 118. The machine learning model is trained on patient data labeled with a respective historic clinical outcome such that the clinical outcome can be predicted based on patient attributes. In some implementations, the in silico clinical study engine 116 predicts the clinical study outcome 118 on combined data of the historic patient data 106 and at least some of the synthetic patient data 114.

The feedback data 112 specifies one or more parameters used in generating the synthetic patient data. The parameters include a sample size, inclusion and exclusion criteria, an algorithmic choice, and additional patient attributes to be included in the patient data. The training engine 110 receives the feedback data 112 and refines the synthetic data generation engine 108, which regenerates the synthetic patient data 114 based on the updated parameters. For example, the feedback data 112 may indicate that the currently set sample size is low for well-powered statistical analysis, and the training engine 110 can increase the sample size for both case and control groups. The synthetic data generation engine 108 uses the increased sample size in regenerating the synthetic patient data 114.

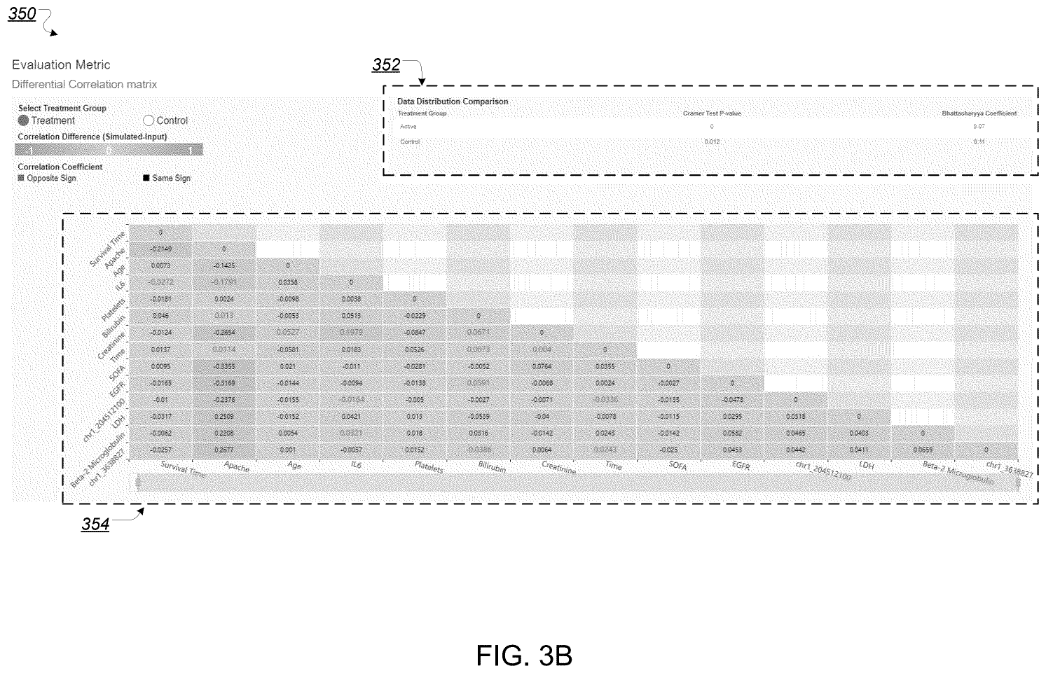

The computer 130 can generate rendering data that, when rendered by a device having a display such as a user device 150 (e.g., a computer having a monitor 150a, a mobile computing device such as a smartphone 150b, or another suitable user device), can cause the device to output data including the clinical study outcome 118. Such rendering data can be transmitted, by the computer 130, to the user device 150 through the network 120 and processed by the user device 150 or associated processor to generate output data for display on the user device 150. In some implementations, the user device 150 can be coupled to the computer 130. In such instances, the rendered data can be processed by the computer 130, and cause the computer 130, on a user interface, to output data that include the clinical study outcome 118. Example user interfaces are described below, referring to FIGS. 3A-3B.

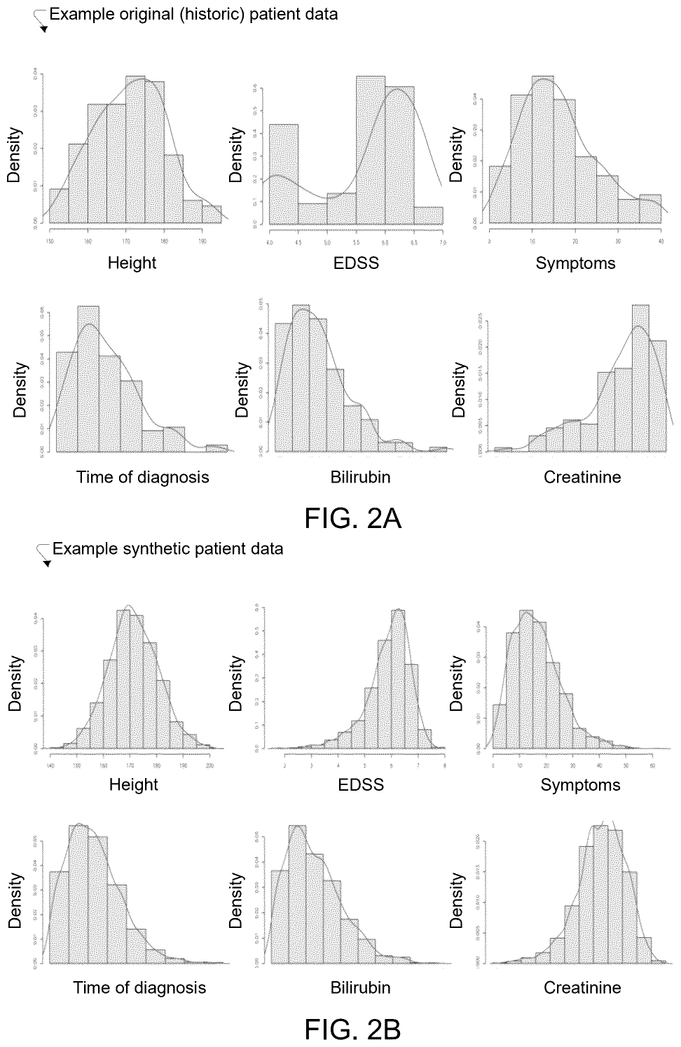

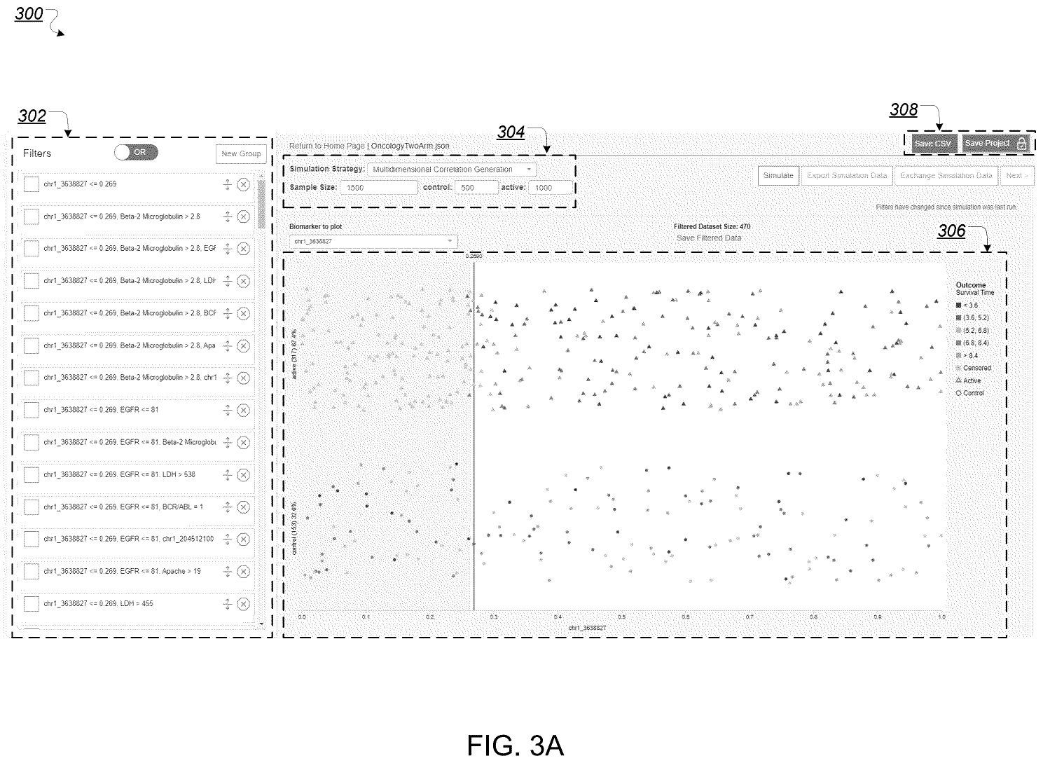

FIG. 2A shows an example of distribution of original (historic) patient data. Each histogram represents distribution of a particular patient attribute. For example, height of patients is distributed with a peak at 175 cm. Patient attributes can be either categorical or continuous. For example, height is continuous, and presence of a disease is categorical. Qualitative metrics can be converted to quantitative values; for example, severity of symptoms may be scored numerically with a higher score indicating more severe symptoms. Referring to FIG. 2A, patient attributes include physiological information (e.g., height), blood test measurements (e.g., creatinine level), molecular measurements (e.g., gene expression), clinical tests (e.g., expanded disability status scale (EDSS)), and medical history information (e.g., time of diagnosis of a disease, symptoms, family history).

FIG. 2B shows an example of distributions of synthetic patient data. The synthetic data generation engine 108 can generate the synthetic patient data. The synthetic patient data reproduce statistical properties from the original patient data. As shown in FIG. 2B, distributions of the synthetic patient data from height to creatinine level are similar to those of the original patient data. The number of patients in the original patient data and the synthetic patient data needs not be same.

FIG. 3A shows an example user interface 300 for displaying a predicted clinical study outcome. In some implementations, the user interface 300 is a web-based user interface displayed on the user device 150, e.g., a smartphone 150b. In some implementations, the user interface 300 is an application loaded on the user device 150, e.g., a server 150c. The user interface 300 includes a filter panel 302, where a user can apply a filter on patient attributes (also referred to as biomarkers). For example, in response to selecting one or more patient attributes, the user interface 300 displays the predicted clinical study outcome stratified by the selected biomarkers in a display panel 306. The display panel 306, in some implementations, displays the predicted clinical study outcome stratified by a group of patients, e.g., case (active) vs. control. Referring to FIG. 3A, a patient survival time is the predicted clinical study outcome. The display panel 306 can use different colors and shapes to represent variations in the predicted clinical study outcome or other filters.

The user interface 300 includes a simulation panel 304, where a user can refine parameters used in generating the synthetic patient data used in predicting the clinical study outcome. In some implementations, the simulation panel 304 includes a case sample size, a control sample size, and an algorithm for generating synthetic patient data. For example, when a user determines that previously generated synthetic patient data are not well-powered due to low sample size, the user may increase the number of samples by inputting the desired sample size in the simulation panel 304. As another example, upon determining that a certain algorithm does not perform well, the user can select a different algorithm to generate synthetic patient data by interacting with the simulation panel 304.

The user interface 300 includes an export panel 308, where a user can select to save the simulation or simulated results, e.g., predicted clinical study outcome for each of simulated data. For example, the user can export the simulation results as a tabular format. The user can also export the result displayed on the display panel 306 as an image.

FIG. 3B shows an example user interface 350. The user interface 350 includes a distribution comparison panel 352 that displays statistical significance on the predicted clinical study outcome between case and control group. For example, the in silico clinical study engine 116 predicts the patient survival time and computes the statistical significance (p-value) between the case group receiving a treatment and the control group not receiving the treatment. Based on the results displayed on the distribution comparison panel 352, a user can refine parameters used in generating the synthetic patient data. The statistical significance, e.g., those displayed on the distribution comparison panel 352, is a p-value corrected for multiple hypotheses. When the user regenerates the synthetic patient data, e.g., after increasing the number of samples or removes outlier data, a number of hypotheses increases, and the statistical significance is recomputed considering the increase in the number of hypotheses to prevent overfitting. The user interface 350 has a correlation panel 354 that displays a multivariate correlation structure among patient attributes. For example, in response to a user selection of case group, the correlation panel 354 displays correlations among the patient attributes in the case group, e.g., a correlation coefficient of 0.0073 between age and survival time and a correlation efficient of −0.0124 between creatinine level and survival time (as shown in FIG. 3B). Based on the multivariate correlation structure, the user can refine samples used in simulating the clinical study. For example, upon determining that gene expression of interleukin 6 (IL6) is highly predictive for patient's survival time, the user may generate additional synthetic patient data across wider range of IL6 gene expression. The user may include additional patient attributes, not included in the current simulation. Continuing the IL6 example, the user may want to include genes co-expressed with IL6, identify historic patient data that include these genes, and generate synthetic patient data based on the historic patient data.

In some implementations, the user interface 300 and the user interface 350 are the same user interface that displays a different view upon a user selection of a desired result.

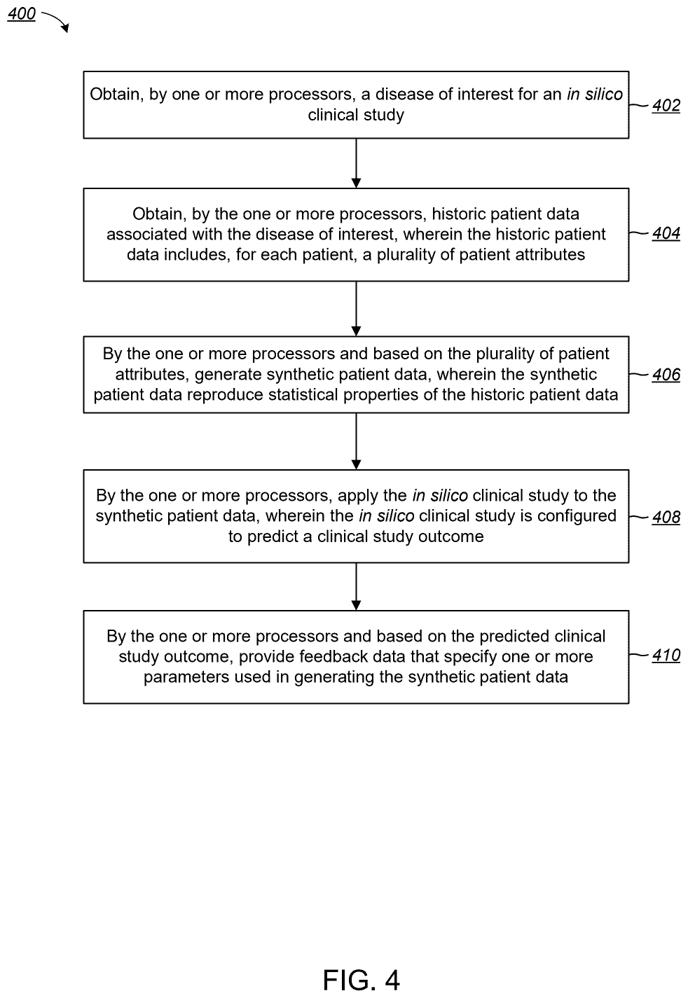

FIG. 4 is a flowchart of an example of a process 400 for generating synthetic patient data and simulating clinical studies. The process will be described as being performed by a system of one or more computers programmed appropriately in accordance with this specification. For example, the computer 130 of FIG. 1 can perform at least a portion of the example process. In some implementations, various steps of the process 400 can be run in parallel, in combination, in loops, or in any order.

The system obtains a disease of interest for an in silico clinical study (402). The disease of interest represents data indicative of the disease of interest, e.g., a file including a name of the disease. The disease of interest needs not be an illness, e.g., colorectal cancer, and includes a condition, e.g., high cholesterol, diabetes, and attention deficit hyperactivity disorder (ADHD). In some implementations, the disease of interest is inputted by a user through a user interface, e.g., by typing a disease of interest “high cholesterol.” In some implementations, the system determines the disease of interest based on a treatment under a clinical study. For example, if the clinical study investigates effectiveness of improving attention and focus levels of patients, the system determines that the disease of interest as ADHD, based in part on a database, e.g., the database 132, which includes knowledge about diseases, e.g., their symptoms and currently available treatments.

The system obtains historic patient data associated with the disease of interest (404). The historic patient data includes, for each patient, a plurality of patient attributes. The historic patient data include a first set of patient data and a second set of patient data. A plurality of patients in the synthetic patient data corresponding to the first set of patient data receives a treatment in the in silico clinical study. To obtain the historic patient data, for example, the system accesses the database 132 that includes encrypted historic patient data 132a and obtain patient data associated with patients with the disease of interest. The obtained historic patient data are divided into case and control groups, where the case group receives a treatment (e.g., antidiabetic drug), and the control group does not. The plurality of patient attributes includes biomarkers of the disease of interest, e.g., age, measurements from a blood test (e.g., creatinine level, LDH level), molecular measurements (e.g., gene expression), and underlying medical conditions related to the disease of interest. In general, the biomarkers of the disease represent variables associated with the disease, e.g., factors that are known to increase or decrease the risk of the disease. In some implementations, the system determines one or more inclusion and exclusion criteria, e.g., having a particular biomarker, and identifies a subset of the historic patient data that meet the inclusion and exclusion criteria. For example, the inclusion and exclusion criteria may identify specific number of case and control patients such that the subset of the historic patient data that meet the criteria is used in generating synthetic patient data.

The system generates, based on the plurality of patient attributes, synthetic patient data (406). The synthetic patient data reproduce statistical properties of the historic patient data. The synthetic patient data need not be limited to continuous (e.g., age) or categorical (e.g., sex) attributes and can encompass both types of data. For the case that the system uses a subset of the historic patient data, the system generates synthetic patient data that correspond to the subset of the historic patient data. In some implementations, to generate the synthetic patient data, the system determines a multivariate correlation structure among the plurality of patient attributes in the historic patient data (e.g., a correlation between mortality and a platelet count of the historic patient data is similar to that of the synthetic patient data) and generates the synthetic patient data that closely maintain the multivariate correlation structure. In some implementations, the system iteratively applies a k-nearest neighbors algorithm until the system generates enough samples of synthetic patient data. For example, the system selects a random sample from the historic patient data and selects k-nearest samples from the random sample, where k indicates the number of patient data to be sampled at a time. Then, the system generates a synthetic sample by computing the average of the k selected samples. The system repeats this process iteratively until it generates required amount of synthetic patient data. In some implementations, the system applies a trained machine learning model to generate synthetic patient data, e.g., a deep learning model trained on a set of historic patient data across diseases. In some implementations, the system applies a transfer learning to a machine learning model that is trained on general data and refines the machine learning model by using domain-specific data, e.g., patient data including patient attributes.

The system applies the synthetic patient data to the in silico clinical study (408). The in silico clinical study is configured to predict a clinical study outcome. The clinical study outcome includes one or more of a treatment response, a disease progression, and an adverse event (e.g., mortality, side effects). In some implementations, the system applies, to the synthetic patient data, a machine learning model trained to predict the clinical study outcome (e.g., a treatment response, a disease progression, and an adverse event) and obtains the predicted clinical study outcome. The machine learning model, e.g., by using convolutional neural networks, is trained on a plurality of training patient data, each labeled with a clinical outcome. In some implementations, the system combines the historic patient data and at least some of the synthetic patient data and applies the in silico clinical study to the combined data. In some implementations, the system displays the clinical study outcome on a user interface, e.g., as shown in FIG. 3A including predicted survival time on the synthetic patient data stratified by case vs. control (case group receiving a treatment) and by one or more patient attributes. The system can compute a statistical significance between the clinical study outcomes between the case and those of the control. As shown in FIG. 3B, the system can determine correlations among patient attributes for case (also referred to as treatment) and control, and based on the correlations, the system can determine if the correlations are significantly different between the group by computing a statistical significance, e.g., a Cramer test p-value and a Bhattacharyya coefficient.

The system provides, based on the predicted clinical study outcome, feedback data that specify one or more parameters used in generating the synthetic patient data (410). The one or more parameters used in generating synthetic patient data include a control sample size, a case sample size, and an algorithm to generate the synthetic patient data. In some implementations, the system identifies one or more biomarkers different from the patient attributes included in the historic patient data, obtains second historic patient data that include the one or more biomarkers, and provides the second historic patient data. The second historic patient data are used to generate second synthetic patient data.

In some implementations, the system provides, on a user interface, the clinical study outcome stratified by the plurality of patient attributes. The user interface includes user selectable elements to adjust a plurality of inclusion and exclusion criteria.

In some implementations, the system validates the synthetic data by comparing a first multivariate correlation structure in the historic patient data and a second multivariate correlation structure in the synthetic patient data. For validation, in some implementations, the system determines a Cramer test p-value and a Bhattacharyya coefficient, where the Cramer test p-value and the Bhattacharyya coefficient are corrected for multiple hypothesis, e.g., the number of iterations in generating the synthetic data that meet the requirement of similarity between the first and the second multivariate correlation structures.

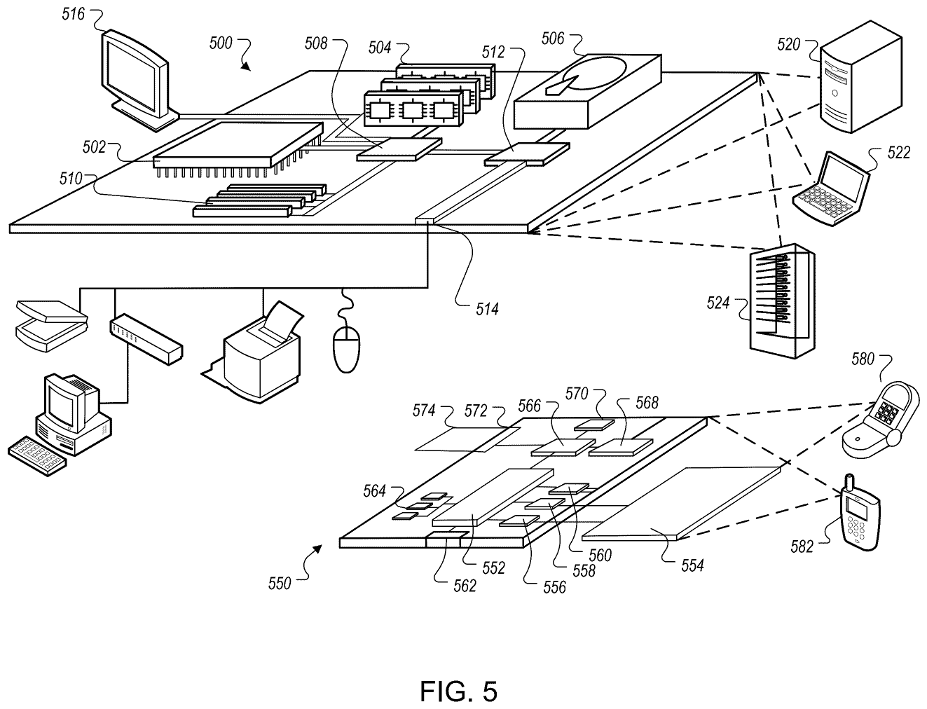

FIG. 5 is an example of a block diagram of system components that can be used to implement a system for generating synthetic patient data and simulating clinical studies.

Computing device 500 is intended to represent various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally, computing device 500 or 550 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be exemplary only, and are not meant to limit implementations of the inventions described and/or claimed in this document.

Computing device 500 includes a processor 502, memory 504, a storage device 506, a high speed controller 508 connecting to memory 504 and high-speed expansion ports 510, and a low speed controller 512 connecting to low speed bus 514 and storage device 506. Each of the components 502, 504, 508, 508, 510, and 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed controller 508. In other implementations, multiple processors and/or multiple buses can be used, as appropriate, along with multiple memories and types of memory. Also, multiple computing devices 500 can be connected, with each device providing portions of the necessary operations, e.g., as a server bank, a group of blade servers, or a multi-processor system.

The memory 504 stores information within the computing device 500. In one implementation, the memory 504 is a volatile memory unit or units. In another implementation, the memory 504 is a non-volatile memory unit or units. The memory 504 can also be another form of computer-readable medium, such as a magnetic or optical disk.

The storage device 506 is capable of providing mass storage for the computing device 500. In one implementation, the storage device 506 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 504, the storage device 506, or memory on processor 502.

The high speed controller 508 manages bandwidth-intensive operations for the computing device 500, while the low speed controller 512 manages lower bandwidth intensive operations. Such allocation of functions is exemplary only. In one implementation, the high speed controller 508 is coupled to memory 504, display 516, e.g., through a graphics processor or accelerator, and to high-speed expansion ports 510, which can accept various expansion cards (not shown). In the implementation, low speed controller 512 is coupled to storage device 506 and low speed bus 514. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

Computing device 550 includes a processor 552, memory 564, and an input/output device such as a display 554, a communication interface 566, and a transceiver 568, among other components. The device 550 can also be provided with a storage device, such as a micro-drive or other device, to provide additional storage. Each of the components 550, 552, 564, 554, 566, and 568, are interconnected using various buses, and several of the components can be mounted on a common motherboard or in other manners as appropriate.

The processor 552 can execute instructions within the computing device 550, including instructions stored in the memory 564. The processor can be implemented as a chipset of chips that include separate and multiple analog and digital processors. Additionally, the processor can be implemented using any of a number of architectures. For example, the processor can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 550, such as control of user interfaces, applications run by device 550, and wireless communication by device 550.

Processor 552 can communicate with a user through control interface 558 and display interface 556 coupled to a display 554. The display 554 can be, for example, a TFT (Thin-Film-Transistor Liquid Crystal Display) display or an OLED (Organic Light Emitting Diode) display, or other appropriate display technology. The display interface 556 can include appropriate circuitry for driving the display 554 to present graphical and other information to a user. The control interface 558 can receive commands from a user and convert them for submission to the processor 552. In addition, an external interface 562 can be provide in communication with processor 552, so as to enable near area communication of device 550 with other devices. External interface 562 can provide, for example, for wired communication in some implementations, or for wireless communication in other implementations, and multiple interfaces can also be used.

The memory 564 stores information within the computing device 550. The memory 564 can be implemented as one or more of a computer-readable medium or media, a volatile memory unit or units, or a non-volatile memory unit or units. Expansion memory 574 can also be provided and connected to device 550 through expansion interface 572, which can include, for example, a SIMM (Single In Line Memory Module) card interface. The expansion memory 574 can provide extra storage space for device 550, or can also store applications or other information for device 550. Specifically, expansion memory 574 can include instructions to carry out or supplement the processes described above, and can include secure information also. Thus, for example, expansion memory 574 can be provide as a security module for device 550, and can be programmed with instructions that permit secure use of device 550. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that can be received, for example, over transceiver 568 or external interface 562.

Device 550 can communicate wirelessly through communication interface 566, which can include digital signal processing circuitry where necessary. Communication interface 566 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through (radio-frequency) transceiver 568. In addition, short-range communication can occur, such as using a Bluetooth, Wi-Fi, or other such transceiver (not shown). In addition, GPS (Global Positioning System) receiver module 570 can provide additional navigation- and location-related wireless data to device 550, which can be used as appropriate by applications running on device 550.

Device 550 can also communicate audibly using audio codec 560, which can receive spoken information from a user and convert it to usable digital information. Audio codec 560 can likewise generate audible sound for a user, such as through a speaker, e.g., in a handset of device 550. Such sound can include sound from voice telephone calls, can include recorded sound, e.g., voice messages, music files, etc. and can also include sound generated by applications operating on device 550.

The computing device 550 can be implemented in a number of different forms, as shown in the figure. For example, it can be implemented as a cellular telephone 580. It can also be implemented as part of a smartphone 582, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1. A computer-implemented method comprising:
obtaining, by one or more processors, a disease of interest for an in silico clinical study;
obtaining, by the one or more processors, historic patient data associated with the disease of interest, wherein the historic patient data includes, for each patient, a plurality of patient attributes;
by the one or more processors and based on the plurality of patient attributes, generating synthetic patient data, wherein the synthetic patient data reproduce statistical properties of the historic patient data;
by the one or more processors, applying the in silico clinical study to the synthetic patient data, wherein the in silico clinical study is configured to predict a clinical study outcome; and
by the one or more processors and based on the predicted clinical study outcome, providing feedback data that specify one or more parameters used in generating the synthetic patient data.

2. The computer-implemented method of claim 1, further comprising:
determining an inclusion and exclusion criterion;
identifying a subset of the historic patient data that meet the inclusion and exclusion criterion; and
generating synthetic patient data that correspond to the subset of the historic patient data.

3. The computer-implemented method of claim 1, wherein the plurality of patient attributes comprises biomarkers of the disease of interest.

4. The computer-implemented method of claim 1, wherein generating the synthetic patient data comprises:
determining a multivariate correlation structure among the plurality of patient attributes in the historic patient data; and
generating the synthetic patient data that maintain the multivariate correlation structure.

5. The computer-implemented method of claim 1, further comprising:
validating, based on comparing a first multivariate correlation structure in the historic patient data and a second multivariate correlation structure in the synthetic patient data, the synthetic patient data.

6. The computer-implemented method of claim 5, wherein comparing the first multivariate correlation structure and the second multivariate correlation structure comprises determining a Cramer test p-value and a Bhattacharyya coefficient, wherein the Cramer test p-value and the Bhattacharyya coefficient are corrected for multiple hypothesis.

7. The computer-implemented method of claim 1, wherein the clinical study outcome comprises one or more of a treatment response, a disease progression, and an adverse event.

8. The computer-implemented method of claim 1, wherein the one or more parameters used in generating synthetic patient data comprise a control sample size, a case sample size, and an algorithm to generate the synthetic patient data.

9. The computer-implemented method of claim 1, further comprising:
providing, on a user interface, the clinical study outcome stratified by the plurality of patient attributes, wherein the user interface comprises user selectable elements to adjust a plurality of inclusion and exclusion criteria.

10. The computer-implemented method of claim 1, wherein the historic patient data include a first set of patient data and a second set of patient data, wherein a plurality of patients in the synthetic patient data corresponding to the first set of patient data receives a treatment in the in silico clinical study.

11. The computer-implemented method of claim 1, wherein applying the in silico clinical study to the synthetic patient data comprises:
applying, to the synthetic patient data, a machine learning model trained to predict the clinical study outcome, wherein the clinical study outcome includes a treatment response, a disease progression, and an adverse event; and
obtaining the predicted clinical study outcome.

12. The computer-implemented method of claim 11, further comprising:
training the machine learning model on a plurality of training patient data, each of the plurality of training patient data is labeled with a clinical outcome, wherein the machine learning model uses convolutional neural networks.

13. The computer-implemented method of claim 1, further comprising:
combining the historic patient data and the synthetic patient data; and
applying the in silico clinical study to the combined historic patient data and the synthetic patient data.

14. The computer-implemented method of claim 1, wherein providing feedback data that specify the one or more parameters used in generating the synthetic patient data comprises:
identifying one or more biomarkers different from the patient attributes included in the historic patient data;
obtaining second historic patient data that include the one or more biomarkers; and
providing the second historic patient data, wherein the second historic patient data are used to generate second synthetic patient data.

15. A system comprising:
one or more processors and one or more storage devices storing instructions that are operable, when executed by the one or more processors, to cause the one or more processors to perform operations comprising:
obtaining, by the one or more processors, a disease of interest for an in silico clinical study;
obtaining, by the one or more processors, historic patient data associated with the disease of interest, wherein the historic patient data includes, for each patient, a plurality of patient attributes;
by the one or more processors and based on the plurality of patient attributes, generating synthetic patient data, wherein the synthetic patient data reproduce statistical properties of the historic patient data;
by the one or more processors, applying the synthetic patient data to the in silico clinical study, wherein the in silico clinical study is configured to predict a clinical study outcome; and
by the one or more processors and based on the predicted clinical study outcome, providing feedback data that specify one or more parameters used in generating the synthetic patient data.

16. The system of claim 15, further comprising:
determining an inclusion and exclusion criterion;
identifying a subset of the historic patient data that meet the inclusion and exclusion criterion; and
generating synthetic patient data that correspond to the subset of the historic patient data.

17. The system of claim 15, wherein generating the synthetic patient data comprises:
determining a multivariate correlation structure among the plurality of patient attributes in the historic patient data; and
generating the synthetic patient data that maintain the multivariate correlation structure.

18. The system of claim 15, wherein the one or more parameters used in generating synthetic patient data comprise a control sample size, a case sample size, and an algorithm to generate the synthetic patient data.

19. The system of claim 15, wherein providing feedback data that specify the one or more parameters used in generating the synthetic patient data comprises:
identifying one or more biomarkers different from the patient attributes included in the historic patient data;
obtaining second historic patient data that include the one or more biomarkers; and
providing the second historic patient data, wherein the second historic patient data are used to generate second synthetic patient data.

20. A non-transitory computer-readable medium, comprising software instructions, that when executed by a computer, cause the computer to execute operations comprising:
obtaining, by the computer, a disease of interest for an in silico clinical study;
obtaining, by the computer, historic patient data associated with the disease of interest, wherein the historic patient data includes, for each patient, a plurality of patient attributes;
by the computer and based on the plurality of patient attributes, generating synthetic patient data, wherein the synthetic patient data reproduce statistical properties of the historic patient data;
by the computer, applying the synthetic patient data to the in silico clinical study, wherein the in silico clinical study is configured to predict a clinical study outcome; and
by the computer and based on the predicted clinical study outcome, providing feedback data that specify one or more parameters used in generating the synthetic patient data.
